(19) United States
(12) Patent Application Publication
(10) Pub. No.: US 2023/0287335 A1
(43) Pub. Date: Sep. 14, 2023

(54) CELL CULTURE MEDIA AND METHODS OF MAKING AND USING THE SAME

(71) Applicant: AMGEN INC., Thousand Oaks, CA (US)

(72) Inventors: Nicholas MARCHAND, Littleton, MA (US); Glen BOLTON, Boston, MA (US); Sara Ann GALL, Longmont, CO (US); Jian WU, Acton, MA (US)

(21) Appl. No.: 18/018,389

(22) PCT Filed: Jul. 27, 2021
(86) PCT No.: PCT/US2021/043289

§ 371 Date: Jan. 27, 2023

Related U.S. Application Data
(60) Provisional application No. 63/058,921, filed on Jul. 30, 2020.

(51) Int. Cl. C12N 5/00; B01D 61/02; B01D 65/08

(52) U.S. Cl. CPC C12N 5/0018; B01D 61/027; B01D 65/08; C12N 2501/105; C12N 2501/33; B01D 2321/168

(57) ABSTRACT

Presented herein are methods for preparing and treating mammalian cell culture media through the addition of low levels of poloxamer and nanofiltration. Low levels of poloxamer improved flux and retained greater levels of a growth factor.

### FIELD

The invention relates to methods for treating mammalian cell culture media through nanofiltering.

### BACKGROUND

Cell culture processes can be used to produce products of therapeutic interest, including recombinant proteins such as Monoclonal antibodies (mAbs). Viral contamination in cell culture processes can result in costly plant shutdown for decontamination. Strategies to avoid viral contamination may include removing or inactivating adventitious viruses in cell culture media prior to production. Nanofiltration can be suitable for various manufacturing processing, including facilities that implement single-use, disposable manufacturing. However, fouling of commercially available nanofilters from cell culture media can result in flux and throughput limitations that lead to relatively high consumable costs. Furthermore, interactions between media components and sterilizing-grade or viral filters could impact media composition, cell culture performance, and ultimately product quality.

### SUMMARY

The present disclosure provides methods of nanofiltering mammalian cell culture media, the method comprising: providing a level of non-ionic surfactant in a quantity of mammalian cell culture media comprising a growth factor, said level of non-ionic surfactant being (i) at least a lower threshold level and (ii) no greater than an upper threshold level, wherein the lower threshold level comprises a level of non-ionic surfactant effective to retain at least 60% of the growth factor following nanofiltration of the culture media, and wherein the upper threshold is effective to permit nanofiltration of the culture media on a nanofilter surface area no more than 40% greater than a nanofilter surface needed to filter the quantity of mammalian cell culture media without non-ionic surfactant; and nanofiltering the quantity of cell culture media, whereby at least 60% of the growth factor is retained following said nanofiltering. In one embodiment, if the quantity of mammalian cell culture media having non-ionic surfactant below the lower threshold level is nanofiltered, at least 40% of the growth factor is lost and/or if quantity of the mammalian cell culture media having non-ionic surfactant above the upper threshold level is nanofiltered, at least 40% greater surface area of nanofilter is needed to nanofilter compared to a quantity of mammalian cell culture media without non-ionic surfactant.

The present disclosure also provides methods of nanofiltering mammalian cell culture media wherein a growth factor is retained in the mammalian cell culture media, the method comprising: providing a level of non-ionic surfactant in a quantity of mammalian cell culture media comprising the growth factor, said level being at least a lower threshold level, wherein the lower threshold level comprises a level of non-ionic surfactant effective to retain at least 60% of the growth factor following nanofiltration of the culture media; and nanofiltering the cell culture media, whereby at least 60% of the growth factor is retained following said nanofiltering. In one embodiment, if the quantity of mammalian cell culture media having non-ionic surfactant above the upper threshold level is nanofiltered, at least 40% greater surface area of nanofilter is needed to nanofilter compared to a quantity of mammalian cell culture media without non-ionic surfactant.

The present disclosure also provides methods of nanofiltering mammalian cell culture media, the method comprising: providing a level of non-ionic surfactant in a quantity of mammalian cell culture media, said level being no more than an upper threshold level, wherein the upper threshold is effective to permit nanofiltration of the culture media on a nanofilter surface area no more than 40% greater than a nanofilter surface needed to filter the quantity of mammalian cell culture media without non-ionic surfactant; and nanofiltering the quantity of cell culture media using a nanofilter. In one embodiment, if the quantity of mammalian cell culture media having non-ionic surfactant below the lower threshold level is nanofiltered, at least 40% of the growth factor is lost.

In certain embodiments of any of the above methods, the level of non-ionic surfactant is greater than or equal to 1 g/L. In certain embodiments, the level of non-ionic surfactant is less than or equal to 5 g/L. In certain embodiments, the level of non-ionic surfactant is 0.1 g/L-5 g/L. In certain embodiments, the level of non-ionic surfactant is 1 g/L-5 g/L.

In certain embodiments of any of the above methods, the non-ionic surfactant comprises poloxamer or poly-vinyl alcohol (PVA). In one embodiment, the non-ionic surfactant comprises poloxamer 188. In one embodiment, the non-ionic surfactant comprises poloxamer 188 and the level of non-ionic surfactant is greater than or equal to 0.1 g/L and less than 1 g/L.

In certain embodiments of any of the above methods, the quantify of mammalian cell culture media is at least 100 L or at least 500 L.

In one embodiment of any of the above methods, the growth factor is IGF-1. In certain embodiments, the growth factor does not comprise insulin.

In certain embodiments of any of the above methods, providing the level of non-ionic surfactant comprises adding non-ionic surfactant to the mammalian cell culture media. In certain embodiments of any of the above methods, providing the level of non-ionic surfactant comprises removing non-ionic surfactant from the mammalian cell culture media.

In certain embodiments of any of the above methods, the mammalian cell culture media is a concentrate. In certain embodiments, the concentrate is at least 2×, 3×, 5×, or 10×. In certain embodiments, the method further comprises diluting the concentrate. In one embodiment, the concentrate is diluted to about 1×.

In certain embodiments of any of the above methods, the mammalian cell culture media comprises mammalian cells and a protein secreted by said mammalian cells prior to said nanofiltering.

In certain embodiments of any of the above methods, the protein comprises a monoclonal antibody.

### BRIEF DESCRIPTION OF THE DRAWINGS

FIGS. 1A-C are graphs showing nanofilter flux vs. loading using cell culture media with varying levels of Pluronic® using (A) Viresolve® barrier filter, (B) ViroSart® HF filter, and (C) Planova™ 20N virus removal filter.

FIGS. 2A-C are graphs showing a filter area requirement (Aₘᵢₙ (m²)) to filter 5000 L of 1× media, or 1000 L of 5× media in 4 h using (A) Viresolve® barrier filter, (B) ViroSart® HF filter, and (C) Planova™ 20N virus removal filter.

FIG. 3 is a graph of filter area requirements (Area (m²)) to filter 5000 L of 1× media, 1× media with 0.1 mg/L IGF-1, or 1× media with 5 mg/L insulin through a Millipore Viresolve® nanofilter in 4 h.

FIGS. 4A-B are graphs showing growth factor levels (A: IGF-1; B: insulin) in media after formulation, sterile-filtration (sterile pool) and nanofiltration (VF pool).

FIG. 5 is a graph of the impact of Pluronic® levels on nanofilter area requirement (for 5000 L batch of 1× media in 4 h) and IGF-1 loss during sterile filtration and viral filtration. The left bars represent the increase in filter area required relative to media without Pluronic® (e.g., if media with Pluronic® required 2× nanofilter area than media without Pluronic®, it would be a 100% increase in Aₘᵢₙ). The right bars represent % of IGF-1 loss during sterile filtration and viral filtration.

### DETAILED DESCRIPTION

Described herein are methods for nanofiltering mammalian cell culture media. It has been observed that during conventional nanofiltration of mammalian cell culture media, filter fouling can occur, leading to increased costs and inhibiting throughput. It has further been observed that conventional nanofiltration can cause loss of growth factors such as IGF-1 from the filtrate. The level of a common shear protectant in cell culture media, Pluronic® F-68 at 0.1% (1 g/L) is a key contributor to nanofilter fouling as well as retention of the critical growth factor IGF-1 on sterilizing-grade filters and nanofilters. By tuning Pluronic® levels in the media, the viral filter throughput and growth factor retention during media filtration can be optimized. As described in Example 2, relatively low levels of shear protectant can cause loss of growth factors such as IGF-1, while relatively high levels of shear inhibitor can cause filter fouling. Thus, in some embodiments, high levels of growth factors such as IGF-1 can be retained during nanofiltering by providing a level of shear protectant in culture media that is at least a lower threshold. In certain embodiments, the lower threshold is 0 g/L, 0.05 g/L, 0.1 g/L or 0.25 10 g/L. In certain embodiments, the amount of shear protectant can be effective to retain at least 60% of the growth factor following nanofiltration of the culture media. In some embodiments, filter fouling can be avoided, inhibited, prevented, or reduced during nanofiltering by providing a level of shear protectant in culture media that is no more than an upper threshold. In certain embodiments, the upper threshold is 25 g/L, 10 g/L, 5 g/L, 1 g/L, 0.9 g/L, 0.8 g/L, 0.7 g/L, 0.6 g/L, or 0.5 g/L. In some embodiments, high levels of growth factors such as IGF-1 can be retained and filter fouling can be avoided, inhibited, prevented, or reduced during nanofiltering by providing a level of shear protectant in culture media that is at least a lower threshold, and no more than an upper threshold. In certain embodiments, the lower threshold is 0 g/L, 0.05 g/L, 0.1 g/L or 0.25 g/L and the upper threshold is 25 g/L, 10 g/L, 5 g/L, 1 g/L, 0.9 g/L, 0.8 g/L, 0.7 g/L, 0.6 g/L, or 0.5 g/L.

Both the foregoing general description and the following detailed description are exemplary and explanatory only and are not restrictive. The use of the singular includes the plural unless specifically stated otherwise. The use of “or” means “and/or” unless stated otherwise. The use of the term “including”, as well as other forms, such as “includes” and “included”, is not limiting. Terms such as “element” or “component” encompass both elements and components comprising one unit and elements and components that comprise more than one subunit unless specifically stated otherwise.

The use of the term “portion” can include part of a moiety or the entire moiety. When a numerical range is mentioned, e.g., 1-5, all intervening values are explicitly included, such as 1, 2, 3, 4, and 5, as well as fractions thereof, such as 1.5, 2.2, 3.4, and 4.1.

“About” or “˜” means, when modifying a quantity (e.g., “about” 3 mM), that variation around the modified quantity can occur. These variations can occur by a variety of means, such as typical measuring and handling procedures, inadvertent errors, ingredient purity, and the like.

“Comprising” and “comprises” are intended to mean that the formulations and methods include the listed elements but do not exclude other unlisted elements. The terms “consisting essentially of” and “consists essentially of,” when used in the disclosed methods include the listed elements, exclude unlisted elements that alter the basic nature of the formulation and/or method, but do not exclude other unlisted elements. A formulation consisting essentially of elements would not exclude trace amounts of other elements, such as contaminants from any isolation and purification methods or pharmaceutically acceptable carriers (e.g., phosphate buffered saline), preservatives, and the like, but would exclude, for example, additional unspecified amino acids. The terms “consisting of” and “consists of” when used to define formulations and methods exclude more than trace elements of other ingredients and substantial method steps for administering the compositions described herein.

Embodiments defined by each of these transition terms are within the scope of this disclosure. “Antibody” or “immunoglobulin” refers to a tetrameric glycoprotein that consists of two heavy chains and two light chains, each comprising a variable domain (V) and a constant domain (C). “Heavy chains” and “light chains” refer to substantially full-length canonical immunoglobulin light and heavy chains; the variable domains (VL and VC) of the heavy and light chains constitute the V region of the antibody and contributes to antigen binding and specificity. “Antibody” includes monoclonal antibodies, polyclonal antibodies, chimeric antibodies, human antibodies, and humanized antibodies. Light chains can be classified as kappa and lambda light chains. Heavy chains are typically classified as mu, delta, gamma, alpha, or epsilon, and define the antibody's isotype as IgM, IgD, IgG, IgA, and IgE, respectively. IgG has several subclasses, including IgG1, IgG2, IgG3, and IgG4. IgM has subclasses including IgM1 and IgM2. IgA is similarly subdivided into subclasses including IgA1 and IgA2. Within full-length light and heavy chains, typically, the variable and constant regions are joined by a “J” region of about 12 or more amino acids, with the heavy chain also including a “D” region of about 10 more amino acids. The variable regions of each light/heavy chain pair typically form the antigen binding site. “Monoclonal antibody” refers to an antibody obtained from a population of substantially homogeneous antibodies, i.e., the individual antibodies comprising the population are identical except for possible naturally occurring mutations that may be present in minor amounts.

“Antibody variants” include antibody fragments and antibody-like proteins with changes to structure of canonical tetrameric antibodies. Typical antibody variants include V regions with a change to the constant regions, or, alternatively, adding V regions to constant regions, optionally in a non-canonical way. Examples include multi-specific antibodies (e.g., bispecific antibodies, trispecific antibodies), antibody fragments that can bind an antigen (e.g., Fab′, F′(ab)2, Fv, single chain antibodies, diabodies), biparatopic and recombinant peptides comprising the forgoing as long as they exhibit the desired biological activity.

Multi-specific antibodies target more than one antigen or epitope. For example, a “bispecific,” “dual-specific”, or “bifunctional” antibody is a hybrid antibody that has two different antigen binding sites. Bispecific antibodies can be produced by a variety of methods including fusing hybridomas or linking Fab′ fragments (Kostelny et al., 1992, J Immunol 148:1547-53; Songsivilai & Lachmann, 1990, Clin Exp Immunol 79:315-21; Wu & Demarest, 2019, Methods 154:3-9). The two binding sites of a bispecific antibody each bind to a different epitope. Likewise, trispecific antibodies have three binding sites and bind three epitopes. Several methods of making trispecific antibodies are known and are being further developed (Wu & Demarest, 2019, Methods 154:3-9; Wu et al., 2018, Protein Eng Des Sel 31:249-256)

“Antibody fragments” include antigen-binding portions of the antibody including, for example, Fab, Fab′, F(ab′)2, Fv, domain antibody (dAb), complementarity determining region (CDR) fragments, CDR-grafted antibodies, single-chain antibodies (scFv), single chain antibody fragments, chimeric antibodies, diabodies, triabodies, tetrabodies, minibody, linear antibody; chelating recombinant antibody, a tribody or bibody, an intrabody, a nanobody, a small modular immunopharmaceutical (SMIP), an antigen-binding-domain immunoglobulin fusion protein, single domain antibodies (including camelized antibody), a VHH containing antibody, or a variant or a derivative thereof, and polypeptides that contain at least a portion of an immunoglobulin that is sufficient to confer specific antigen binding to the polypeptide, such as one, two, three, four, five or six CDR sequences, as long as the antibody retains the desired binding activity.

The terms “flux” or “flow rate” refer to the rate at which of fluid passes through a filtration medium of a given area and is defined as the amount of permeate produced per unit area of membrane surface per unit time. A flux unit can be expressed as L/hr/m² or LMH.

“Fouling” of a membrane refers to the deposition of a particle on a membrane surface or membrane pores whereby the flow of liquids through the membrane's pore is restricted.

### Cell Culture Media

The methods of the present disclosure can be used for the preparation of any cell culture media containing a protein where loss of the protein occurs during filtration in the absence of or with low levels of a shear protectant. For example, it has been discovered that a minimum level of Pluronic® is needed to retain the growth factor IGF-1. While the same effect was not seen with insulin, it is likely that other proteins could be lost in the absence of a shear protectant. Accordingly, the methods disclosed herein could be used for the filtration of all culture media where it is desired to retain one or more proteins in the cell culture media.

“Cell culture” or “culture” refer to the growth and propagation of cells outside of a multicellular organism or tissue. Suitable culture conditions for mammalian cells are described, for example, in Animal cell culture: A Practical Approach, D. Rickwood, ed., Oxford University Press, New York (1992). Mammalian cells may be cultured in suspension or while attached to a solid substrate.

As used herein, the terms “cell culture medium” (which may also be referred to herein as “culture medium,” “cell culturing media,” “tissue culture media,” and variations of these root terms) have their ordinary and customary meaning as would be understood by one of ordinary skill in the art in view of this disclosure. They refer to any nutrient solution used for growing cells, e.g., animal or mammalian cells, and which generally provides at least one or more components from the following: an energy source (usually in the form of a carbohydrate such as glucose); one or more of all essential amino acids, and generally the twenty basic amino acids, plus cysteine; vitamins and/or other organic compounds typically required at low concentrations; lipids or free fatty acids; and trace elements, e.g., inorganic compounds or naturally occurring elements that are typically required at very low concentrations, usually in the micromolar range.

The nutrient solution may optionally be supplemented with additional components to optimize growth of cells, such as hormones and other growth factors, for example insulin growth factor-1 (IGF-1), insulin, transferrin, epidermal growth factor, serum, and the like; salts, such as calcium, magnesium and phosphate, and buffers, e.g., HEPES; nucleosides and bases, such as adenosine, thymidine, hypoxanthine; and protein and tissue hydrolysates, such as hydrolyzed plant or animal protein (peptone or peptone mixtures, which can be obtained from animal byproducts, purified gelatin or plant material); antibiotics, such as gentamycin; polyamines, such as putrescine, spermidine and spermine (see International Patent Application Publication No. WO 2008/154014) and pyruvate (see U.S. Pat. No. 8,053,238), anti-apototic compounds, e.g., MDL 28170, cypermethrin, cyclosporine A, BBMP, Bongkrekic acid, S-15176 difumerate, cyclic pifithrin-a, pifithrin mu, BI-6C9, NSCI, NS3694 or Necrostatin-1 (see International Patent Application Publication No. WO 2014/022102) depending on the requirements of the cells to be cultured and/or the desired cell culture parameters. In certain embodiments, the cell culture media contains IGF-1.

Cell culture media include those that are typically employed in and/or are known for use with any cell culture process, such as, but not limited to, batch, extended batch, fed-batch and/or perfusion or continuous culturing of cells.

A “base” (or batch) cell culture medium or feed medium refers to a cell culture medium that is typically used to initiate a cell culture and is sufficiently complete to support the cell culture.

A “growth” cell culture medium or feed medium refers to a cell culture medium that is typically used in cell cultures during a period of exponential growth, a “growth phase”, and is sufficiently complete to support the cell culture during this phase. A growth cell culture medium may also contain selection agents that confer resistance or survival to selectable markers incorporated into the host cell line. Such selection agents include, but are not limited to, geneticin (G418), neomycin, hygromycin B, puromycin, zeocin, methionine sulfoximine, methotrexate, glutamine-free cell culture medium, cell culture medium lacking glycine, hypoxanthine and thymidine, or thymidine alone.

A “production” cell culture medium or feed medium refers to a cell culture medium that is typically used in cell cultures during the transition when exponential growth is ending and during the subsequent transition and/or production phases when protein production takes over. Such cell culture medium is sufficiently complete to maintain a desired cell density, viability and/or product titer during this phase.

A “perfusion” cell culture medium or feed medium refers to a cell culture medium that is typically used in cell cultures that are maintained by perfusion or continuous culture methods and is sufficiently complete to support the cell culture during this process. Perfusion cell culture medium formulations may be richer or more concentrated than base cell culture medium formulations to accommodate the method used to remove the spent medium. Perfusion cell culture medium can be used during both the growth and production phases.

Cell culture medium components, including a shear protectant, may be completely milled into a powder medium formulation; partially milled with liquid supplements added to the cell culture medium as needed; or added in a completely liquid form to the cell culture.

Cell cultures can be supplemented with concentrated feed medium containing components, such as nutrients and amino acids, which are consumed during the course of the production phase of the cell culture. Concentrated cell culture medium can contain some or all of the nutrients necessary to maintain the cell culture; in particular, concentrated medium can contain nutrients identified as or known to be consumed during the course of the production phase of the cell culture. Concentrated medium may be based on just about any cell culture media formulation. Concentrated feed medium can contain some or all the components of the cell culture medium at, for example, about 2×, 3×, 4×, 5×, 6×, 7×, 8×, 9×, 10×, 12×, 14×, 16×, 20×, 30×, 50×, 100×, 200×, 400×, 600×, 800×, or even about 1000× of their normal amount.

Cell cultures can also be supplemented with independent concentrated feeds of particular nutrients which may be difficult to formulate or are quickly depleted in cell cultures. Such nutrients may be amino acids such as tyrosine, cysteine and/or cystine (see e.g., International Patent Application Publication No. 2012/145682). For example, a concentrated solution of tyrosine can be independently fed to a cell culture grown in a cell culture medium containing tyrosine, such that the concentration of tyrosine in the cell culture does not exceed 8 mM. In another example, a concentrated solution of tyrosine and cystine is independently fed to the cell culture being grown in a cell culture medium lacking tyrosine, cystine or cysteine. The independent feeds can begin prior to or at the start of the production phase. The independent feeds can be accomplished by fed batch to the cell culture medium on the same or different days as the concentrated feed medium. The independent feeds can also be perfused on the same or different days as the perfused medium. Such independent feeds can be added to the cell culture medium after one or more days, and can also be added repeatedly during the course of the production phase, as long as tyrosine, cysteine and cystine depletion in the cell culture medium is avoided.

Cell culture methods can be employed to continuously feed a mammalian cell culture, such as those that do not employ feedback control (see International Patent Application Publication No. WO 2013/040444).

Cell culture medium, in some embodiments, is serum-free and/or free of products or ingredients of animal origin. Cell culture medium, in some embodiments, is chemically defined, where all of the chemical components are known.

Animal or mammalian cells may be cultured in a medium suitable for the particular cells being cultured. Commercially available media can be utilized in nanofiltration methods of some embodiments, herein. Examples of commercially available media include, but are not limited to, Iscove's Modified Dulbecco's Medium, RPMI 1640, Minimal Essential Medium-alpha. (MEM-alpha), Dulbecco's Modification of Eagle's Medium (DMEM), DME/F12, alpha MEM, Basal Medium Eagle with Earle's BSS, DMEM high Glucose, with Glutamine, DMEM high glucose, without Glutamine, DMEM low Glucose, without Glutamine, DMEM:F12 1:1, with Glutamine, GMEM (Glasgow's MEM), GMEM with glutamine, Grace's Complete Insect Medium, Grace's Insect Medium, without FBS, Ham's F-10, with Glutamine, Ham's F-12, with Glutamine, IMDM with HEPES and Glutamine, IMDM with HEPES and without Glutamine, IP41 Insect Medium, 15 (Leibovitz)(2×), without Glutamine or Phenol Red, 15 (Leibovitz), without Glutamine, McCoy's 5A Modified Medium, Medium 199, MEM Eagle, without Glutamine or Phenol Red (2×), MEM Eagle-Earle's BSS, with glutamine, MEM Eagle-Earle's BSS, without Glutamine, MEM Eagle-Hanks BSS, without Glutamine, NCTC-109, with Glutamine, Richter's CM Medium, with Glutamine, RPMI 1640 with HEPES, Glutamine and/or Penicillin-Streptomycin, RPMI 1640, with Glutamine, RPMI 1640, without Glutamine, Schneider's Insect Medium or any other media known to one skilled in the art, which are formulated for particular cell types. To the foregoing exemplary media can be added supplementary components or ingredients, including optional components, in appropriate concentrations or amounts, as necessary or desired, and as would be known and practiced by those having in the art using routine skill.

In certain embodiments, the cell culture media is of a sufficient volume for a production bioreactor. In a related embodiment the production bioreactor has a capacity of at least 500 L. In a related embodiment the production bioreactor has a capacity of at least 500 L, at least 2000 L or up to 20,000 L or more. In a related embodiment the production bioreactor has a capacity of at least 1000 L to 2000 L.

### Shear Protectants

In the nanofiltering methods described herein, the cell culture medium may comprise one or more shear protectants including non-ionic surfactants. Examples of non-ionic surfactants include, but are not limited to, polyvinyl alcohol, polyethylene glycosl, and non-ionic block copolymer surfactants. Also included are alkyl poly(ethylene oxide), copolymers of poly(ethylene oxide) and poly(propylene oxide) (EO-PO block copolymers), poly(vinylpyrrolidone), alkyl polyglucosides (such as sucrose monostearate, lauryl diglucoside, or sorbitan monolaureate, octyl glucoside and decyl maltoside), fatty alcohols (cetyl alcohol or olelyl alcohol), or cocamides (cocamide MEA, cocamide DEA and cocamide TEA).

Also included are block copolymers based on ethylene oxide and propylene oxide, also referred to as polyoxypropylene-polyoxyethylene block copolymers. These molecules are nonionic triblock copolymers having a central hydrophobic chain of polyoxypropylene (poly(propylene oxide)) flanked by two hydrophilic chains of polyoxyethylene (poly(ethylene oxide)). Of particular interest are those having 70 polyoxypropylene units and 30 units of each of the polyoxyethylene chains. In a preferred embodiment the block copolymer is poloxamer 188 (CAS #90003-11-6 with an average molecular weight of 8.4 kD, BASF Chemical, Washington, N.J.) which is sold under various brand names such as Pluronic® F68, Kolliphor® P-188, Lutrol® F68, and Lutror 188. These polyoxypropylene-polyoxyethylene block copolymers may protect cells from bubble-induced death due to sparging and foam in the reactor.

In nanofiltering methods of some embodiments, the non-ionic surfactant comprises, consists essentially of, or consists of poloxamer (such as poloxamer 188) or polyvinyl alcohol. In nanofiltering methods of some embodiments, the non-ionic surfactant comprises, consists essentially of, or consists of poloxamer (such as poloxamer 188). In certain embodiments, the amount of shear protectant is at least 0 g/L, 0.05 g/L, 0.1 g/L or 0.25 g/L (which may be considered a lower threshold). In certain embodiments, the amount of shear protectant is no more than 25 g/L, 10 g/L, 5 g/L, 1 g/L, 0.9 g/L, 0.8 g/L, 0.7 g/L, 0.6 g/L, or 0.5 g/L (which can be considered an upper threshold).

### Nanofilters and Nanofiltering

As used herein, “nanofilter” has its ordinary and customary meaning as would be understood by one of ordinary skill in the art in view of this disclosure. It refers to a filter suitable for excluding viruses from the filtrate. Nanofiltering can employ ultrafiltration membranes which may be formed from polysulfone, polyarylsulphones, cellulose, cellulose acetate, polyether sulfone, polyurethane, poly (urea urethane), polybenzimidazole, polyimide, polyamide, polyetherimide, polyacrylonitrile, poly(ethylene terephthalate), polypropylene, polyaniline, poly(ethylene oxide), poly(ethylene naphthalate), poly (butylene terephthalate), styrene butadiene rubber, polystyrene, poly(vinyl chloride) , poly(vinyl alcohol), poly(vinylidene fluoride), poly(vinyl butylene), copolymers, derivative compounds and blends thereof, and combinations thereof. Representative ultrafiltration membranes include, but are not limited to Viresolve® membranes, Viresolve® Pro membranes, Viresolve® 180 membranes, Viresolve® 70 membranes, Viresolve® NFP membranes, Viresolve® NFR membranes, Retropore™ membranes, Virosart® CPV membranes, Planova™ 75 membranes, Planova™ 35 membranes, Planova™ 20 membranes, Planova™ 15N membranes, VAG 300 membranes, Ultipor® DVD membranes, Ultipor® DV50 membranes, Ultipor® DV20 membranes, and DVD Zeta Plus VR™ filters. A typical nanofilter may permit at least 4, 5, or 6 log reduction value (LRV) of viruses in cell culture media.

“Log reduction value” (LRV) is a measurement of filtration retention efficiency that is equivalent to the ratio of the log of the challenge concentration divided by the filtrate concentration (LRV=Log₁₀ Challenge/Filtrate). A challenge concentration refers to the concentration of viral materials in the cell culture media. A filtrate (i.e., cell culture media) is considered to be sterile if it has an LRV of at least 4.85, and filtrates having LRVs of between 6 and 7 are preferred. In one embodiment of the invention, a log reduction value of greater than or equal to 4.85 is obtained following the treatment of cell culture media with nanofiltration. In another embodiment, a log reduction value of between 6 and 7 is obtained following the treatment of cell culture media with nanofiltration.

Without being limited by theory, a typical virus may have a dimeter of 20 nm (e.g., for a non-enveloped virus) to 50 nm (e.g., for an enveloped virus). By way of example, a suitable nanofilter for nanofiltration methods described herein may comprise, consist essentially of, or consist of polyethersulfone (PES). By way of example, a suitable nanofilter for nanofiltration methods described herein may have an effective pore size of 22 nm or less, 20 nm or less, for example 20 nm or less, 18 nm or less, 15 nm or less, 12 nm or less, 10 nm or less, 5 nm or less, 2 nm or less, or 1 nm or less, including ranges between any two of the listed values, for example, 1-20 nm, 1-15 nm, 1-10 nm, 2-20nm, 2-15 nm, 2-10 nm, 5-20 nm, 5-15 nm, 5-10 nm, 10-20 nm, or 10-15 nm.

Filtration can be effected with one or more ultrafiltration membranes either by dead end (normal) flow filtration (NFF) or by tangential flow filtration (TFF). In some embodiments of the present invention normal flow filtration is used. “Normal flow filtration:, used interchangeably with the terms “dead end”, “single pass”, and “direct flow filtration”, refers to a virus filter filtration process wherein the liquid flow path is directed usually perpendicular to the filter surface, dependent on the construction of the filter module the fluid stream could also be directed tangential to the filter membrane. However, in contrast to crossflow filtration, no recirculation of retentate is applied, which means that the specific flow rate before and after the filter is identical. In NFF the feed stream is passed through a membrane and the large molecular weight substances are trapped in the filter while the filtrate is released at the other end. In one embodiment of the invention tangential flow filtration may be performed in the method according to the invention. In the context of the present invention “tangential flow filtration,” is used interchangeably herein with the term “crossflow filtration.” In tangential flow mode, the liquid flow path on the upstream side of the filter is directed roughly parallel to or tangential to or across the filter surface. Passage of the permeate is facilitated by restricting the flow of retentate relative to feed, resulting in backpressure to the system and permitting permeate migration through the filter membrane. The constant sweeping current across the membrane surface has the effect of minimizing clogging by contaminants in the product being filtered. In TFF, the majority of the feed flow travels tangentially across the surface of the filter, rather than into the filter. As such, the filter cake is substantially washed away during the filtration process, increasing the length of time that a filter unit can be operational. Ultrafiltration membranes for either mode of filtration can be supplied in either a cartridge (NFF) form, such as VIRESOLVE® NFP viral filters, or as cassettes (for TFF), such as PELLICON® cassettes. Examples of suitable nanofiltration techniques are described, for example, in Liu et al., 2000, Biotechnol. Prog. 16:425-434. By way of example, a cell culture medium may be passed through a nanofilter by applying pressure to the cell culture medium. It will be appreciated that the rate of filtration may depend up on the pressure applied to the cell culture medium, and the surface area of the filter. It will further be appreciated that as more cell culture media is filtered through a nanofilter, a nanofilter may begin to foul. To conserve resources, and for efficiency of workflow, it will be appreciated that it is advantageous for a relatively small surface area of nanofilter to be capable of nanofiltering a cell culture media.

The filtration is performed at a temperature from about 2° C. to about 60° C., or about 10° C. to about 50° C., preferably about 30° C. to about 45° C. In one embodiment, the lower limit of the temperature range is about 2° C., about 4° C., about 8° C., about 10° C., about 15° C., about 20° C., about 22° C., about 25° C., about 30° C., about 37° C. or about 40° C. The upper limit of the temperature range is about 10° C., about 20° C., about 22° C., about 25° C., about 30° C., abut 37° C., about 40° C., about 50° C., or about 60° C. In one embodiment, the temperature is in a range from about 4° C. to about 45° C., or at a temperature range from about 10° C. to about 40° C., or from about 20° C. to about 40° C., or from about 30° C. to about 37° C. The filtration is performed at a pressure between 0 and 80 psi, for example, between 0 and 50 psi.

The cell culture medium can be treated using additional methods or devices to sterilize or disinfect media prior to addition to a bioreactor and/or cell culture. In one embodiment, the cell culture media is treated using high temperature short time (HTST) (see, e.g., U.S. Pat. No. 7,420,183). In one embodiment, the cell culture media is treated using UV in combination with filtration (see, e.g., International Patent Application Publication Nos. WO 2008/157247; WO 2012/115874; WO 2013/063298 and WO 2013/138159). In another embodiment, the cell culture media is treated with chemicals that inactivate viruses, such as solvents, detergents, psoralen, or beta-propiolactone.

As those of ordinary skill in the art would appreciate, all embodiments of the invention can be implemented with the aid of any available system technically useful for the purpose, e.g. a variable-speed or fixed-speed peristaltic pump, a centrifugal pump, etc. Any kind of pressurized vessel or other container can be used to generate flow through the virus filter with constant or variable pressure during the filtration process. Those of ordinary skill in the art will appreciate that the choice of filter type and mode (dead end filtration or tangential flow filtration) will depend on factors such as composition, the protein content, the molecular weight distribution, impurity/particulate load or any other biochemical or physical property in the feed to be processed, process requirements and limitations (allowable pressure, process time, volumes to be filtered) or characteristics of the potential viral contaminant, e.g. virus size. Availability of an in-process integrity test and logistics of viral clearance studies must also be taken into consideration. Dead end filtration should typically be employed for feed streams of high purity to yield a reasonable process flux whereas in some embodiments tangential flow filtration can accommodate feed streams with high particulate load. In some preferred embodiments normal flow filtration is preferred in combination with a continuous filtration mode using a least one virus filter having an effective pore size of maximum 75 nm.

In some embodiments, the filtrate or the flow of the filtrate obtained from the filtration process is fed to a large-scale cell culture and bioreactor respectively. A “large-scale” cell culture, as used herein, refers to a cell culture at a scale of at least about 100 L, at least about 200 L, at least about 300 L, at least about 400 L, at least about 500 L, at least about 1000 L, at least about 1500 L, at least about 2000 L, at least about 2500 L, at least about 3000 L, at least 4000 L, at least about 5000 L, at least about 7500 L, at least about 10000 L or at least about 20000 L. In a preferred embodiment the filtrate flow obtained in any method according to the invention is fed to a bioreactor used in a chemostat process, a perfusion process or a fed batch process, preferably by continuous filtration.

In the methods disclosed herein, lower levels of a shear protectant than commonly used may be employed in cell culture processes. If lower levels are employed it may be desirable to add additional shear protectant post-nanofiltration to a suitable amount. In such embodiments, the final amount of shear protectant is at least 1 g/L, 5 g/L, or 10 g/L.

The treated cell culture media can be used to support the growth of a number of different cell types. In one embodiment of the invention, the treated cell culture media is used to support the growth of mammalian cells. In another embodiment of the invention, the mammalian cells are capable of producing antibodies. In yet another embodiment of the invention, the treated cell culture media is used to support the growth of insect cells.

### REFERENCES

Chisti, 2000, Trends Biotechnol 18:420-432

Gigout et al., 2008, Biotechnol Bioeng 100:975-987

Hu et al., 2008, Biotechnol Bioeng 101:119-127

Tharmalingam et al., 2008, Mol Biotechnol 39:167-177

Tharmalingam et al., 2015, Biotechnol Bioeng 112:832-7

Xu et al., 1995, Chin J Biotechnol 11:101-107

Clincke et al., 2011, Biotechnol Prog 27: 181-190

The Examples that follow are illustrative of specific embodiments of the invention, and various uses thereof. They are set forth for explanatory purposes only and are not to be taken as limiting the invention.

### EXAMPLES

### Example 1

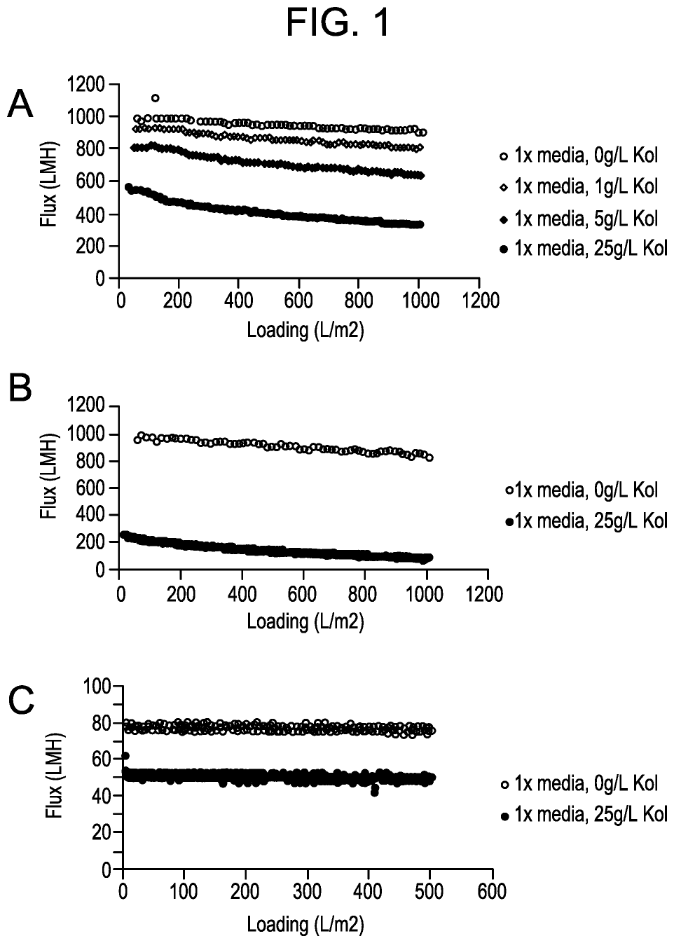

The effect of Pluronic® on filter fouling was investigated. Commercially available media (GE HyClone™ DME/F12+2.5 mM L-Glutamine+15 mM HEPES powder) was prepared with different concentrations of Pluronic® (tradename Kolliphor®; 0, 1, 5, or 25 g/L) using a 20% solution. The media was sterile filtered and applied to one of three commercial nanofilters (Millipore Virosolve® Barrier (VBarrier), Sartorius ViroSart® MF, Asahi Planova™ 20N) using a PendoTECH Filter Screening System with a Pump Module (PendoTECH, Princeton, N.J.). The flux was calculated in real time using a PendoTECH Filter Screening system. See FIG. 1. Fouling was observed on all 3 filters at all Pluronic® concentrations tested as indicated by decreased flux during loading compared to media with no Pluronic®. The media without added Pluronic® demonstrated minimal fouling with all 3 nanofilters tested as indicated by higher flux during loading.

The improved flux and throughput observed when using media with lower levels of Pluronic® translates to increased filter efficiency, lower required filter area per L of media, and thus lower operational costs.

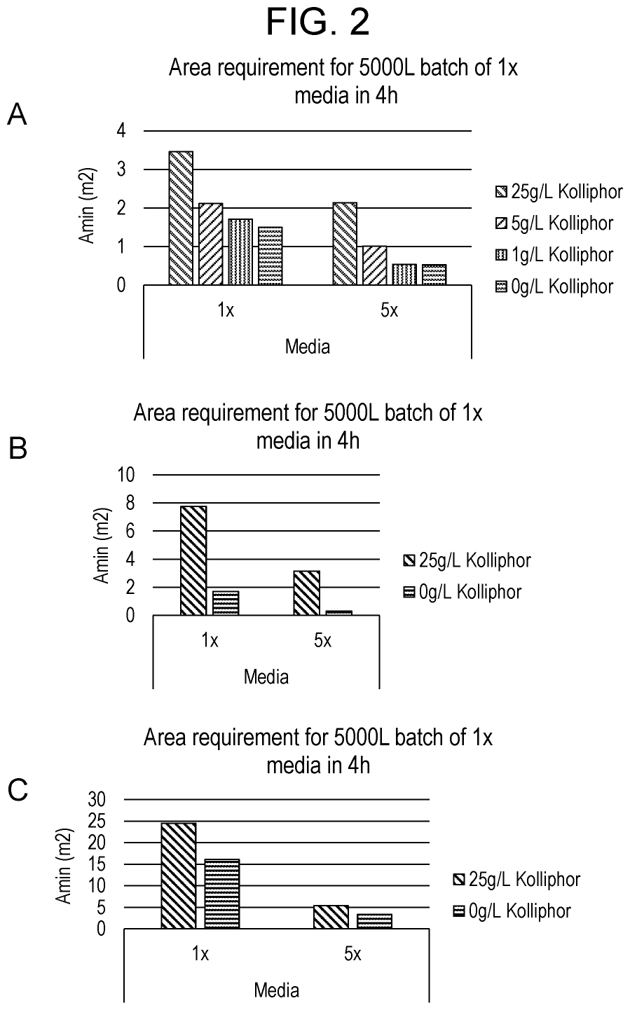

Removing Pluronic® to improve filter efficiency as measured by filter area requirement was tested at varying media concentrations. Media was prepared at 1× as described above and in a concentrated form at 5× and subjected to filtration as described above using the same three filters described above. FIG. 2 shows calculated filter area requirements to filter a 5000 L batch of 1× media in 4 h or a 1000 L batch of 5× media in 4 h. Area requirement was calculated as follows:

Aₘᵢₙ=(Vbatch/Vmax)+(Vbatch/(Jo*tmax))
Vbatch is the batch volume (5000 or 1000)
Vmax is the maximum throughput as calculated by the PendoTECH system from a plot of Time/Volume (h/L) vs Time (h)
Jo is the initial flux as calculated by the PendoTECH system from a plot of Time/Volume (h/L) vs Time (h)
tmax is the batch time (4 h)

1× media and 5× concentrated media without Pluronic® leads to improved filter efficiency as shown by the lowest area requirements. See FIG. 2. Using media concentrates and omitting Pluronic® can provide improved filter efficiency beyond that of using either technique alone. See FIG. 2. Preparing media in a concentrated form and diluting it to its target concentration just prior to use can be an effective strategy to decrease batch time, facility footprint, and in some cases required filtration area.

These experiments suggest that while shear protectants are critical components in cell culture media, they can be added back into the media formulation after nanofiltration and could go through a separate form of viral risk mitigation such as high pH inactivation, or autoclaving.

### Example 2

While the data is Example 1 suggests the omission of Pluronic® from cell culture prior to filtration would be a good strategy to overcome filter fouling and maximize the efficiency, cell cultures utilizing this strategy did not perform as well as those containing Pluronic® as evidenced by low growth (data not shown). The impact of nanofiltration on growth factor levels in the media was evaluated by filtering 1× media containing varying levels of Pluronic® and one of two critical growth factors, IGF-1 or insulin. Growth factor retention over both sterilizing-grade filters (Sartorius Sartopore 2 XLM 0.2/0.1 pm) and nanofilters (Millipore VBarrier) was studied.

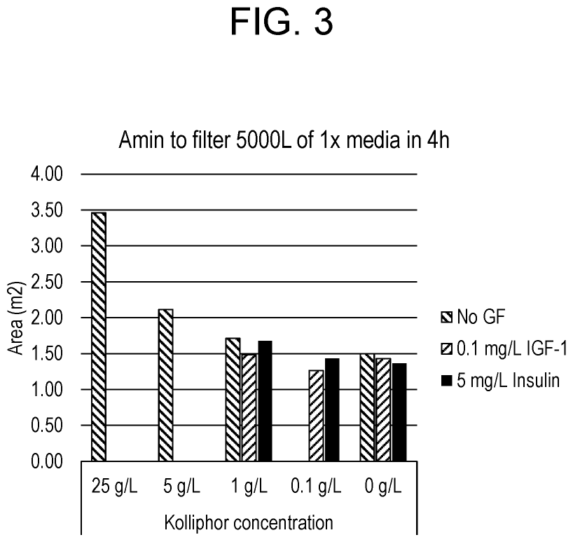

Culture media was formulated as described above with no Pluronic®, 0.1 g/L or 1 g/L Pluronic®. The media was further formulated with IGF-1 (0.1 ml/L) or insulin (5 mg/L), filtered through the Sartopore filter using the PendoTECH system and then filtered through the VBarrier filter again using the PendoTECH system. Area requirements were calculated as described in Example 1. At low concentrations of Pluronic®, no significant difference in nanofilter fouling was seen when comparing media without a growth factor to media containing growth factors. See FIG. 3.

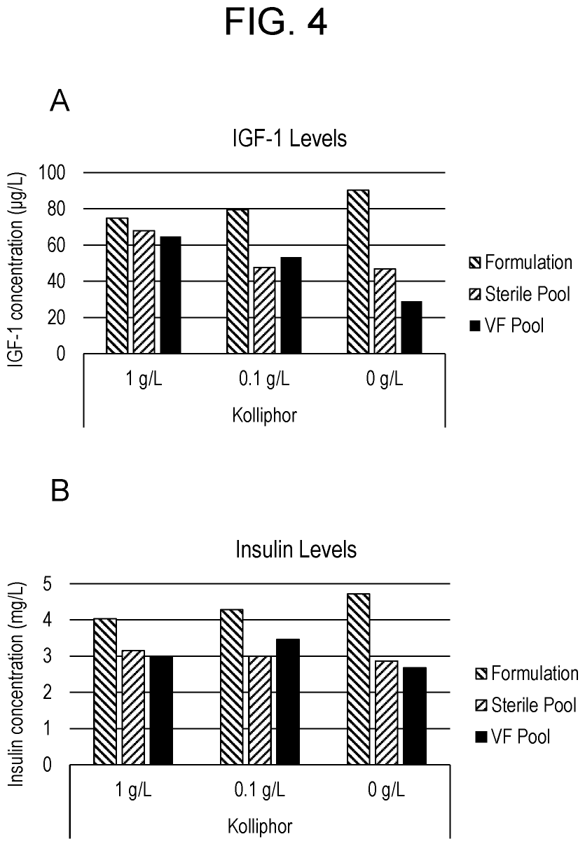

However, all samples containing growth factors demonstrated some growth factor loss during sterile and/or nanofiltration. IGF-1 and insulin levels were determined by solid phase sandwich ELISA with the appropriate antibody (biotinylated anti-recombinant human IGF-1, IGF-1 affinity-purified goat IgG, R&D Systems, Inc., Minneapolis, Minn. or biotinylated mouse monoclonal anti-human insulin antibody, Millipore Sigma, St. Louis, Mo.). See FIG. 4. Target IGF-1 levels were 100 μg/L and target insulin levels were 5 mg/L. Interestingly, the presence of Pluronic® in the media improved IGF-1 retention during sterile and nanofiltration. Of further note, the same pattern was not observed from experiments on media containing insulin. It is surprising and novel that Pluronic® keeps the growth factor IGF-1 in solution. This was not expected and not observed with a second growth factor (insulin).

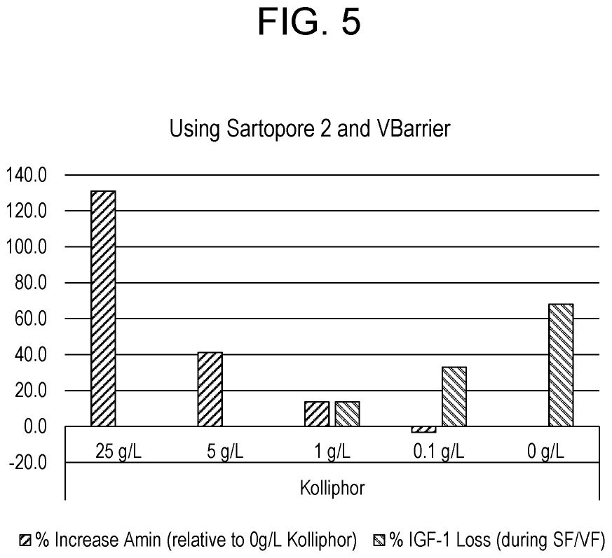

Nanofilter fouling and retention of the critical growth factor IGF-1 can both be optimized by controlling the concentration of a shear protectant (in this case Pluronic®) in the cell culture media. Filter efficiency and Pluronic® levels were optimized to minimize IGF-1 loss for a 5000 L batch of 1× media in 4 h. IGF-1 loss was measured during sterile and viral filtration as described above. See FIG. 5. The bars on the left represent the % increase in filter area required relative to media without Pluronic® (e.g. if media with Pluronic® required 2× nanofilter area than media without Pluronic®, it would be a 100% increase in Amin). The bars on the right represent % of IGF-1 lost during sterile and viral filtration.

1. A method of nanofiltering mammalian cell culture media, the method comprising:
providing a level of non-ionic surfactant in a quantity of mammalian cell culture media comprising a growth factor, said level of non-ionic surfactant being (i) at least a lower threshold level and (ii) no greater than an upper threshold level,
wherein the lower threshold level comprises a level of non-ionic surfactant effective to retain at least 60% of the growth factor following nanofiltration of the culture media, and
wherein the upper threshold is effective to permit nanofiltration of the culture media on a nanofilter surface area no more than 40% greater than a nanofilter surface needed to filter the quantity of mammalian cell culture media without non-ionic surfactant; and
nanofiltering the quantity of cell culture media, whereby at least 60% of the growth factor is retained following said nanofiltering.

2. The method of claim 1,
wherein if the quantity of mammalian cell culture media being nanofiltered has non-ionic surfactant below the lower threshold level, at least 40% of the growth factor is lost, and/or
wherein if the quantity of mammalian cell culture media being nanofiltered has non-ionic surfactant above the upper threshold level, at least 40% greater surface area of nanofilter is needed to nanofilter the culture media compared to a quantity of mammalian cell culture media without non-ionic surfactant.

3. A method of nanofiltering mammalian cell culture media wherein a growth factor is retained in the mammalian cell culture media, the method comprising:
providing a level of non-ionic surfactant in a quantity of mammalian cell culture media comprising the growth factor, said level being at least a lower threshold level,
wherein the lower threshold level comprises a level of non-ionic surfactant effective to retain at least 60% of the growth factor following nanofiltration of the culture media; and
nanofiltering the cell culture media, whereby at least 60% of the growth factor is retained following said nanofiltering.

4. The method of claim 3, wherein if the quantity of mammalian cell culture media being nanofiltered has non-ionic surfactant above the upper threshold level, at least 40% greater surface area of nanofilter is needed to nanofilter the culture media compared to a quantity of mammalian cell culture media without non-ionic surfactant

5. A method of nanofiltering mammalian cell culture media, the method comprising:
providing a level of non-ionic surfactant in a quantity of mammalian cell culture media, said level being no more than an upper threshold level,
wherein the upper threshold is effective to permit nanofiltration of the culture media on a nanofilter surface area no more than 40% greater than a nanofilter surface needed to filter the quantity of mammalian cell culture media without non-ionic surfactant; and
nanofiltering the quantity of cell culture media using a nanofilter.

6. The method of claim 5, wherein if the quantity of mammalian cell culture media being nanofiltered having has non-ionic surfactant below the lower threshold level, at least 40% of the growth factor is lost.

7. The method of claim 1, wherein the non-ionic surfactant comprises poloxamer or poly-vinyl alcohol (PVA).

8. The method of claim 7, wherein the non-ionic surfactant comprises poloxamer 188, and wherein the level of non-ionic surfactant is greater than or equal to 0.1 g/L and less than 1 g/L.

9. The method of claim 1, wherein the quantify quantity of mammalian cell culture media is at least 500 L.

10. The method of claim 1, wherein the growth factor is IGF-1.

11. The method of claim 1, wherein providing the level of non-ionic surfactant comprises adding non-ionic surfactant to the mammalian cell culture media.

12. The method of claim 1, wherein providing the level of non-ionic surfactant comprises removing non-ionic surfactant from the mammalian cell culture media.

13. The method of claim 1, wherein the mammalian cell culture media is a concentrate.

14. The method of claim 13, wherein the concentrate is at least 2×, 3×, 5×, or 10×.

15. The method of claim 13, further comprising diluting the concentrate.

16. The method of claim 15, wherein the concentrate is diluted to about lx.

17. The method of claim 1, wherein the protein comprises a monoclonal antibody.
